(19) United States
(12) Patent Application Publication
(10) Pub. No.: US 2023/0330402 A1
(43) Pub. Date: Oct. 19, 2023

(54) A SWALLOWABLE CAPSULE DEVICE

(71) Applicant: Novo Nordisk A/S, Bagsvaerd (DK)

(72) Inventors: Jorrit Jeroen Water, Frederiksberg (DK); Brian Mouridsen, Fredensborg (DK); Drago Sticker, Malmoe (SE); Aghiad Ghazal, Koebenhavn SV (DK); Adam Jun Bohr, Frederiksberg (DK); Jacob Pyung Hwa Jepsen, Frederiksberg (DK); Peter Herskind, Hilleroed (DK); Cody Edward Cleveland, Koebenhavn K (DK); Jens Lolle Laursen, Jyllinge (DK)

(21) Appl. No.: 18/025,363

(22) PCT Filed: Sep. 29, 2021
(86) PCT No.: PCT/EP2021/076892

§ 371 Date: Mar. 8, 2023

(30) Foreign Application Priority Data
Sep. 30, 2020 (EP) 20199488.6

(51) Int. Cl. A61M 31/00

(52) U.S. Cl. CPC A61M 31/002

(57) ABSTRACT

A capsule device (10) suitable for swallowing into a lumen of an intestinal tract of a patient comprises a housing section (12), a delivery outlet (20) for jet injection by a delivery assembly (22), an expansion assembly (26) and an expansion control mechanism (30). The expansion section (28) is arranged laterally opposite to and physically separated from the delivery outlet (20). An expansion section (28) of the expansion assembly (26) is laterally expandable from a non-expanded configuration to an expanded configuration for positioning the delivery outlet (20) against the lumen wall (14). The expansion control mechanism (30) is configured to activate the expansion assembly (26) under a predetermined condition to permit change of the expansion section (28) from the non-expanded configuration to the expanded configuration.

This invention relates to a capsule device suitable for swallowing into a lumen of an intestinal tract of a patient.

Devices for oral administration are attractive for numerous drug delivery applications including delivery of biomacromolecules, such as proteins and other biologics, to the gastrointestinal (GI) tract. Such oral administration is likely to increase patient compliance, reduce drug administration costs and improve the therapeutic outcomes compared with the more invasive forms of drug administration, e.g. subcutaneous injection. One of the challenges with oral-GI administration is that the drug must be delivered at the desired location in the GI tract and ensuring that sufficient drug deposition occurs in the tissue to provide relevant drug absorption can be difficult. An important part of sufficient drug deposition is ensuring proximity of the oral administration device to the correct part of the GI tract, e.g. the intestinal wall, when drug delivery takes place.

Existing jet injector systems for jet drug delivery are known in the art. US 2016/0228646 A1 discloses a particle delivery device, in particular hand-held device, configured to collimate particles entrained in a gas flow stream and to focus the particles such that a beam of particles is perpendicular to a tissue surface.

WO 2020/106,750 A1 include disclosure of ingestible devices that are configured to dispense a dispensable substance in form of a liquid. In most variants the substance is dispensed as liquid jets from a plurality of nozzles directed perpendicular to a longitudinal axis of the ingestible device, e.g. in a manner wherein the nozzles are uniformly distributed relative to a circumference of the ingestible device. The parameter “Jet stable length” is used to refer to the distance from a nozzle opening that a dispensable substance delivered through the opening remains in the form of a jet. During trans-epithelial delivery, the fluid jet has a jet stable length that is sufficient for the fluid jet to travel across a nozzle stand-off distance to reach the interface of the lumen of the GI tract and the surface of the GI tract facing the lumen.

Having regard to the above, it is an object of the present invention to provide a swallowable capsule device which is improved with respect to therapeutic substance delivery and therapeutic substance depot formation inside the lumen wall.

According to a first aspect of the invention there is provided capsule device suitable for swallowing into a lumen of an intestinal tract of a patient, the lumen having a lumen wall, the capsule device comprising:
a housing section shaped as an elongated object extending along an axis, the housing section defining an interior and having an exterior surface, the interior being configured to contain a therapeutic substance in use,
a delivery outlet disposed at the housing section and laterally to the axis, the delivery outlet being configured to allow needle-less jet injection of the therapeutic substance into the lumen wall,
wherein the capsule device further comprises an expansion assembly comprising an expansion section arranged laterally opposite to and physically separated from the delivery outlet, wherein the expansion section is laterally expandable from a non-expanded configuration to an expanded configuration for positioning the delivery outlet against the lumen wall, and
wherein the capsule device comprises an expansion control mechanism configured to activate the expansion assembly under a predetermined condition to permit change of the expansion section from the non-expanded configuration to the expanded configuration.

The delivery outlet being configured to allow needle-less jet injection means that the capsule device is suitable for this type of injection into the lumen wall. The delivery outlet may be provided in the form of a jet nozzle.

In some embodiments, the capsule device may further include a delivery assembly that includes a jet injector configured to deliver, in use, the therapeutic substance through the delivery outlet into the lumen wall to penetrate an intestinal mucosal lining of the lumen wall by jet injection. Such a delivery assembly including a jet injector allows for needle-less jets to deliver a dose of a desired therapeutic substance, e.g. an active pharmaceutical ingredient (API) to the lumen wall. In this way, the ingestible capsule device does not include sharp needle points and a mechanism which actuates and retracts the needle is also not required. Moreover, the needle-less jet injection is believed to reduce the pain and/or trauma at the injection site compared to a needle delivery.

Existing jet injector systems for jet delivery are known in the art. A skilled person would understand how to select an appropriate jet injector that provides the correct jetting power to deliver the therapeutic substance into the lumen wall, for example from WO 2020/106,750 (PROGENITY INC). Further details and examples are provided further on in the application.

In solution of the prior art, when a dose of the therapeutic substance is expelled by means of a jet nozzle, a pronounced recoil effect is likely to occur which acts to move the jet nozzle away from the targeted tissue area. For example, for capsule devices wherein a single jet nozzle arrangement is located at an end of the housing section, the recoil effect will cause a torque to be exerted onto the capsule device with the result that the jet stream is moved sideways in the course of the expelling action. Hence, for trans-epithelial delivery wherein an initial penetration has been made into a target tissue area by a first portion of the dose, the remaining portion of the dose will be directed towards other tissue areas, potentially leading to penetration of an enlarged tissue area, or potentially leading to loss of therapeutic substance into the lumen of the intestinal tract.

In accordance with the invention, the inclusion of an expansion assembly with an expansion section which is laterally expandable from a non-expanded configuration to an expanded configuration for positioning the delivery outlet against the lumen wall allows for the capsule to be oriented correctly within the intestinal tract ready for jet injection of the therapeutic substance. Further the expansion section serves to fixate the delivery outlet relative to the targeted tissue area during the jet injection process. Thus, the expandable section ensures both proximity of the delivery outlet to the injection site but also ensures that the full dose is delivered through the same penetration opening as initially established at the initiation of the jet delivery action. Moreover, the expansion section assuming a non-expanded configuration allows for ease of swallowing the capsule device for the patient.

The expansion section being laterally opposite and physically separate from the delivery outlet firstly helps achieve correct orientation of the capsule device when the expansion section assumes the expanded configuration. Secondly, in particular the physical separation feature, means that the expansion action and delivery action do not interfere with one another which might otherwise cause one or both to fail. It also means that the two actions can be carried out separately from one another, if desired, and in response to separate triggers and are not reliant on one another.

Meanwhile, the inclusion of an expansion control mechanism means that the expansion section assumes the expanded configuration in response to a predetermined condition such that expansion occurs at the desired location within the GI tract of the patient. The predetermined condition may include one or more of a predetermined time after ingestion of the capsule device, a predetermined location in the GI tract, one or more characteristics of the GI tract (e.g. pH, pressure, acidity, temperature, etc.).

In some embodiments, a single delivery outlet is disposed at the housing section. For example, the single delivery outlet may define a jet nozzle which is arranged along a first side portion of the capsule housing section and at a particular axial location thereof, and wherein the expansion section is disposed at a second side portion of the capsule housing section opposing the first side portion. In different variants, the delivery outlet may be arranged at one end of the housing section, or may be disposed at an axial position in between the two opposing ends, e.g. midways between the two end-portions of the housing section.

In other embodiments, a plurality of delivery outlets, e.g. jet nozzles, are disposed at the housing section in a manner wherein one or more expansion sections are arranged laterally opposite to and physically separated from the delivery outlets.

In certain embodiments of the capsule device, in use, the therapeutic substance forms a liquid drug substance that is expelled through the drug outlet, e.g. through a jet nozzle as a liquid jet stream.

In alternative embodiments of the capsule device, the therapeutic substance is provided in the form of particles, and wherein collimate particles are entrained in a gas flow stream through the drug outlet, e.g. through a jet nozzle as a particle jet stream.

The expansion section may comprise an inflatable balloon, wherein the balloon assumes a non-inflated configuration when the expansion section assumes the non-expanded configuration and assumes an inflated configuration when the expansion section assumes the expanded configuration. The inflatable balloon may form an inflatable bag, wherein the bag assumes a folded configuration when the expansion section assumes the non-expanded configuration and assumes an inflated configuration when the expansion section assumes the expanded configuration.

It will be understood that by the capsule device being configured to be swallowable into the lumen it is considered to be ingestible, i.e. it can be swallowed whole.

The use of an inflatable balloon and/or an inflatable bag has the advantage that the inflated size and pressure of the expansion section can be controlled and selected so that it is suitable for the intended use. Since the pressure of the expansion section onto the lumen wall may be the cause of discomfort to the patient, it is important that the size and pressure of the expansions section can be controlled. The inflatable bag/balloon is of the type that maintains a defines a set volume or shape once inflated so that the pressure exerted by the balloon or bag on the lumen wall is controlled. In this way, the bag/balloon may be made from a non-stretchable material so that it cannot continue to inflate beyond the desired volume or shape.

Optionally, the expansion section comprises a swellable component or substance configured to expand when exposed to gastrointestinal fluid. The swellable component or substance may comprise a hydrogel configured to expand from the non-expanded configuration to the expanded configuration when exposed to gastrointestinal fluid.

The use of a hydrogel provides a means of creating an expanded configuration without the need for a complex mechanism to trigger such expansion.

Preferably, the swellable component or substance may comprise a sponge material configured to expand from the non-expanded configuration to the expanded configuration when exposed to gastrointestinal fluid.

The soft and cushioned nature of sponge material helps to reduce pressure on the lumen wall when the expansion section pushes against the lumen wall, and therefore reduces discomfort to the patient. Moreover, the more a sponge swells the less outward force it will apply, which also helps to reduce any pressure on the lumen wall. The use of sponge material is also simple and reliable (i.e. exposure to fluid causes expansion).

In embodiments where a swellable component is used, the swellable component is preferably chosen such that it has a controlled maximum expansion so that the pressure on the lumen wall can be controlled.

The sponge material, when in the expanded configuration, may create a passage therethrough.

Configuring the sponge material (e.g. via its shape and/or how it is attached to the capsule) so that it creates a passage when expanded helps to prevent blockages in the GI tract. For example, it allows passage of chyme through the intestinal tract. Moreover, the passage is created immediately on expansion of the sponge material and so blockages are reduced throughout the delivery procedure.

Optionally, the inflatable balloon contains a plurality of swellable components or substances configured to expand when exposed to fluid.

The combination of swellable components and an inflatable balloon (or bag, as the case may be) provides a reliable means of expansion via the swellable components (e.g. sponge material) while being able to control and/or restrict the ultimate shape and/or size of the expansion section via the balloon. The pressure exerted on the lumen wall will be controlled by the characteristics of the chosen swellable material inside the balloon, which can be done in a reliable manner. Moreover, the swellable components can be made small enough to pass safely through the remainder of the GI tract after substance delivery.

Preferably, the inflatable balloon may include at least one aperture to permit exposure of the swellable components or substances to gastrointestinal fluid.

The inclusion of an aperture in the inflatable balloon to expose the swellable components to gastrointestinal fluid provides a reliable means of activating expansion of the swellable components, when it is desired to do so, without the need for another internal mechanism to cause such activation.

In one embodiment, the inflatable balloon is configured to release the swellable component or substances after predetermined time.

Such an arrangement helps to prevent blockages in the GI tract by essentially disposing of the expansion section. For example, this could be done by making the swellable components small enough to pass safely through the reminder of the GI tract, forming them from biodegradable material (which may biodegrade after being exposed fully to the intestinal fluid), forming the balloon so that it dissolves after a predefined time period, configuring the balloon to release from the capsule so that it too can pass safely through the tract. The balloon may release the swellable components by opening or dissolving after predetermined time (which may be related to the conditions within the tract).

Optionally, the expansion control mechanism includes an effervescent reaction mechanism configured to activate an effervescent reaction to inflate the balloon to assume the expanded configuration.

Including an effervescent reaction to inflate the balloon to assume the expanded configuration means that the size and pressure of the balloon can be accurately controlled by selecting the correct amount of effervescent material.

The expansion control mechanism may include a separator which separates components that, when mixed, cause the effervescent reaction, the separator being configured to dissolve when exposed to gastrointestinal fluid to allow the components to mix.

Such a separator allows triggering of the effervescent reaction based on the conditions of GI tract, which means correct timing of the reaction can be achieved.

The expansion section may be configured to break up into separate expansion section pieces after a predefined amount of time of being exposed to gastrointestinal fluid. In addition, or alternatively, the expansion section may be configured to break away from the housing section after a predefined amount of time of being exposed to gastrointestinal fluid.

Such breaking up or breaking away helps to clear the lumen of obstruction.

In some embodiments, the expansion control mechanism includes a coating configured to dissolve when exposed to gastrointestinal fluid.

The use of such a coating prevents activation of the expansion control mechanism until the capsule is in the correct part of the GI tract, i.e. the intestine, thus preventing premature expansion of the expansion assembly. The coating may be used in combinations with other triggers or expansion control mechanisms, such as the dissolvable separator as outlined above.

In some variants of the capsule device, the delivery assembly that includes the jet injector also comprises a trigger arrangement for initiating jet injection through the drug outlet. In some forms the trigger arrangement is provided to comprise an environmentally-sensitive mechanism.

In some forms, the capsule device is configured for swallowing by a patient and travelling into a lumen of a GI tract of a patient, such as the small intestine or the large intestine, respectively.

The environmentally-sensitive mechanism may in certain embodiments be a GI tract environmentally-sensitive mechanism. The GI tract environmentally-sensitive mechanism may comprise a trigger member, wherein the trigger member is characterised by at least one of the group comprising:
a) the trigger member comprises a material that degrades, erodes and/or dissolves due to a change in pH in the GI tract;
b) the trigger member comprises a material that degrades, erodes and/or dissolves due to a pH in the GI tract;
c) the trigger member comprises a material that degrades, erodes and/or dissolves due to a presence of an enzyme in the GI tract; and
d) the trigger member comprises a material that degrades, erodes and/or dissolves due to a change in concentration of an enzyme in the GI tract.

In alternative forms, the trigger arrangement may also be or include an electronic trigger.

Preferred embodiments of the invention will now be described, by way of non-limiting examples, with reference to the accompanying drawings in which:

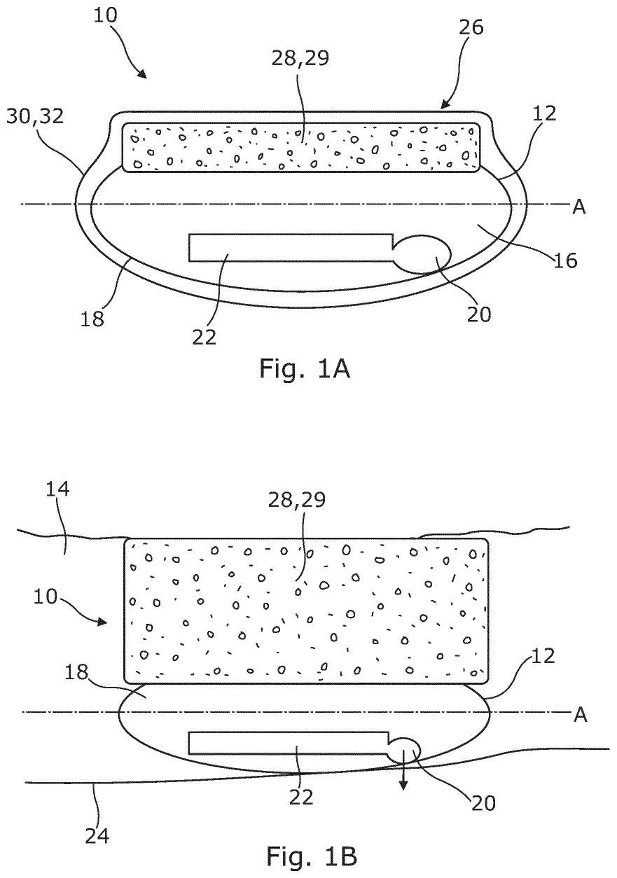

FIG. 1a shows a capsule device according to a first embodiment of the invention with the expansion section in the non-expanded configuration;

FIG. 1b shows the capsule device of FIG. 1a with the expansion section in the expanded configuration in vivo;

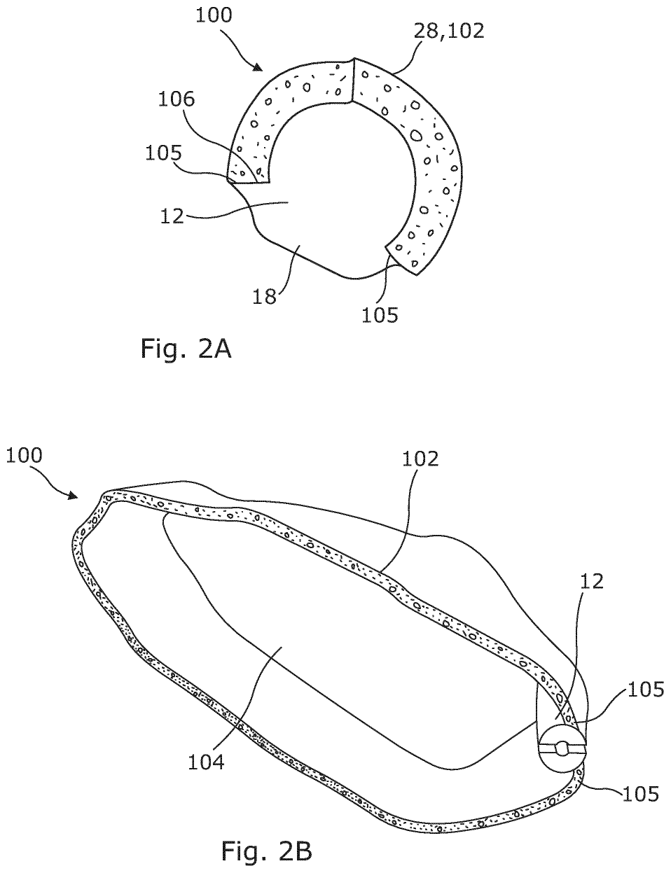

FIG. 2a shows a capsule device according to a second embodiment of the invention with the expansion section in the non-expanded configuration;

FIG. 2b shows the capsule device of FIG. 2a with the expansion section in the expanded configuration;

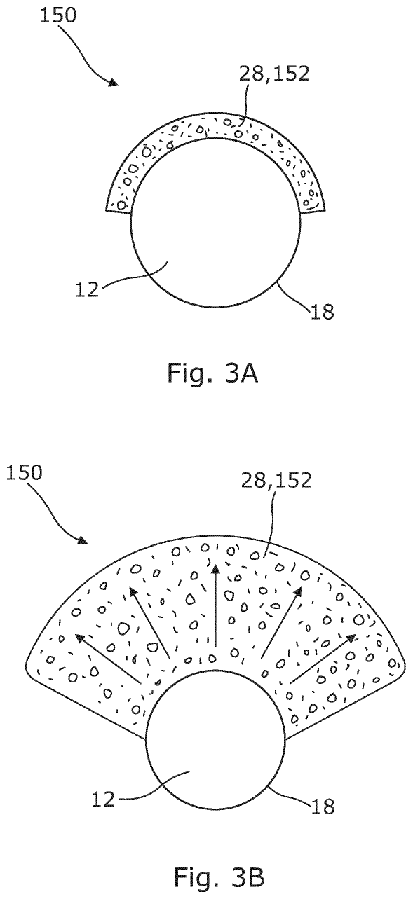

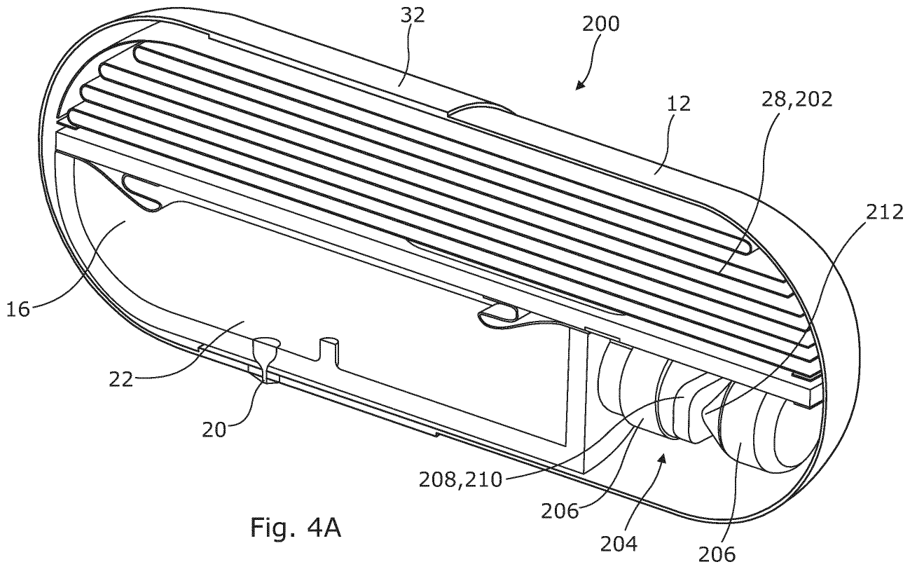

FIG. 3a shows a capsule device according to a third embodiment of the invention with the expanded section in the non-expanded configuration;

FIG. 3b shows the capsule device of FIG. 3a with the expanded section in the expanded configuration;

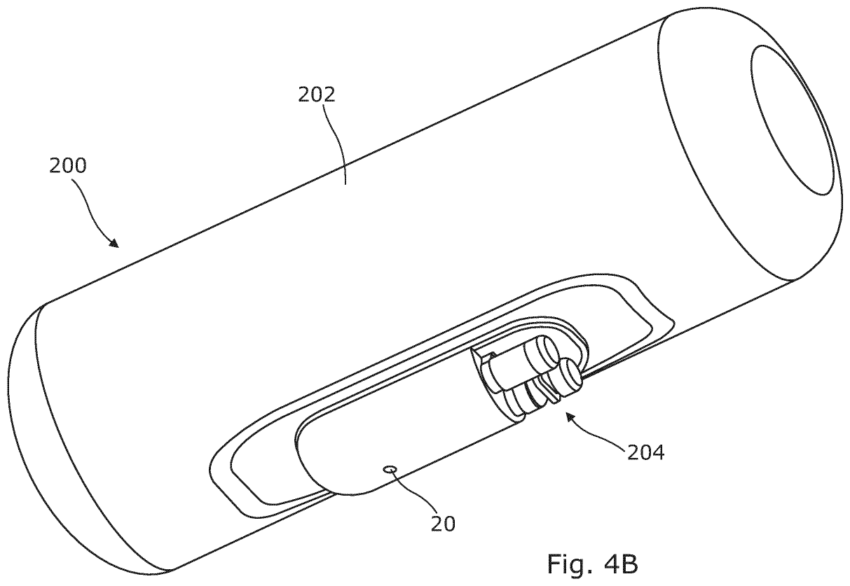

FIG. 4a shows a capsule device according to a fourth embodiment of the invention with the expanded section in the non-expanded configuration;

FIG. 4b shows the capsule device of FIG. 4a with the expanded section in the expanded configuration;

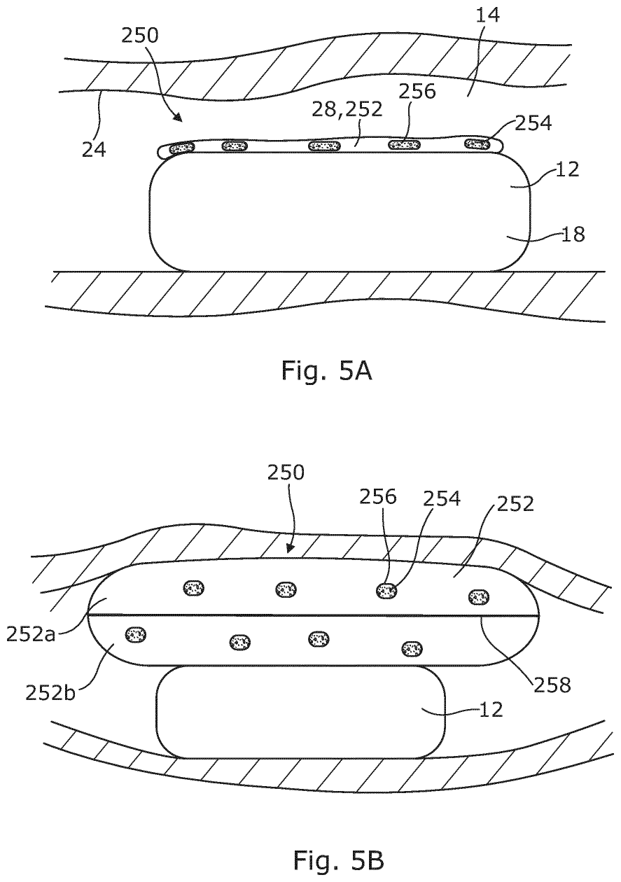

FIG. 5a shows a capsule device according to a fifth embodiment of the invention with the expanded section in the non-expanded configuration;

FIG. 5b shows the capsule device of FIG. 5a with the expanded section in the expanded configuration;

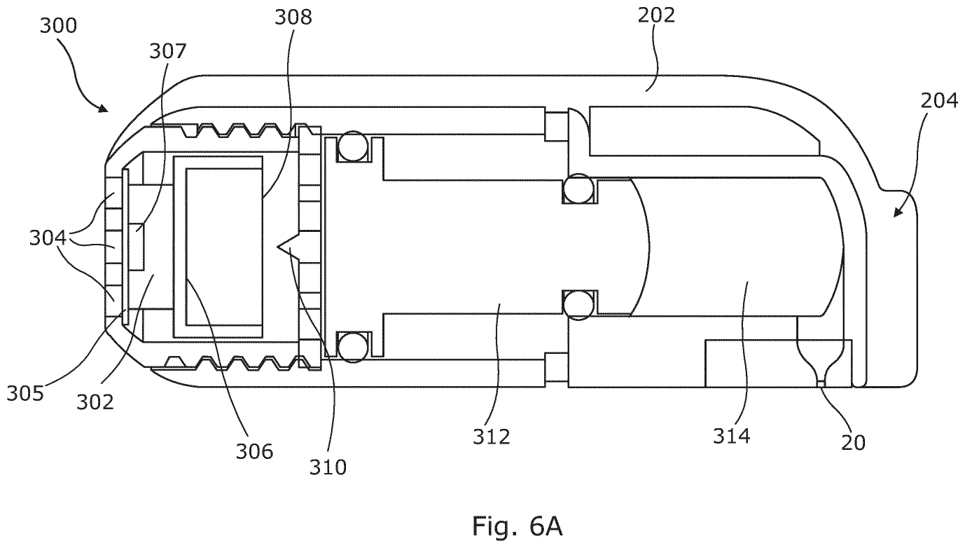

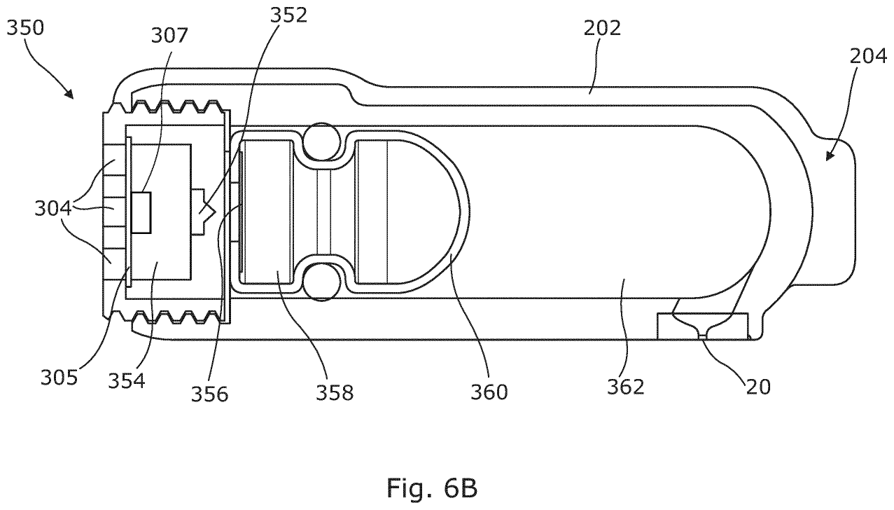

FIG. 6a shows a capsule device according to a sixth embodiment of the invention;

FIG. 6b shows a capsule device according to a seventh embodiment of the invention;

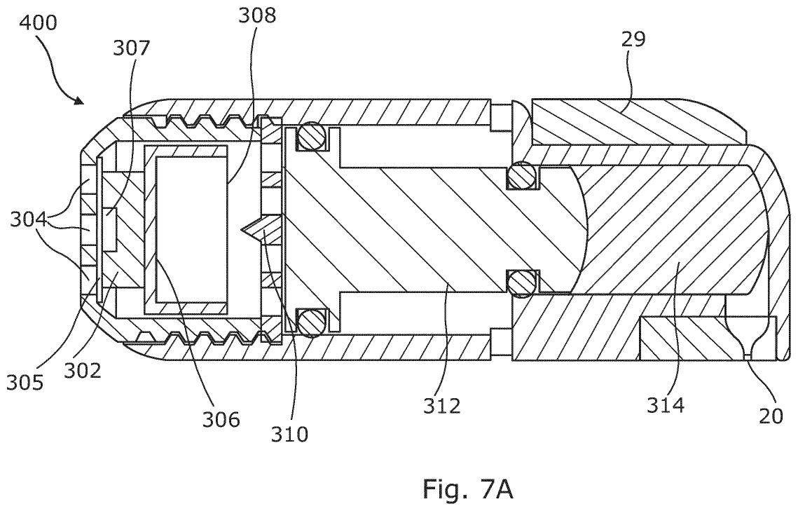

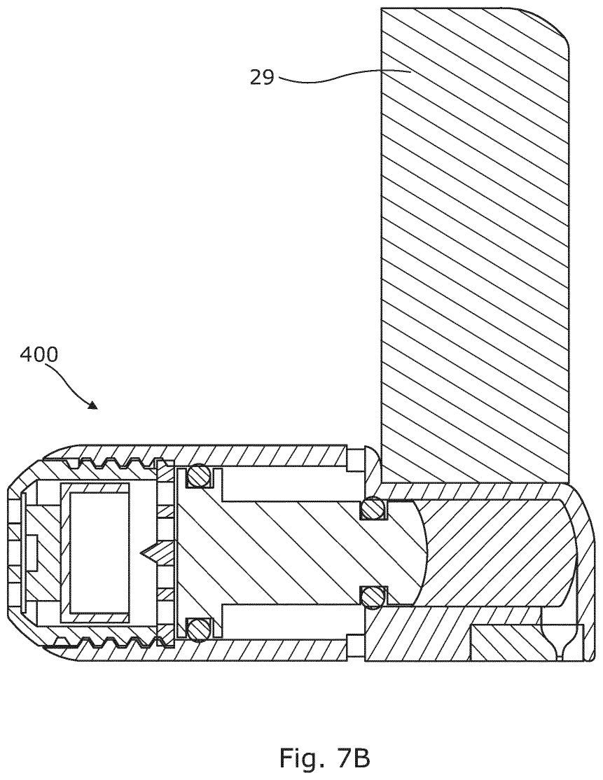

FIGS. 7a, 7b and 7c show a capsule device according to an eighth embodiment of the invention;

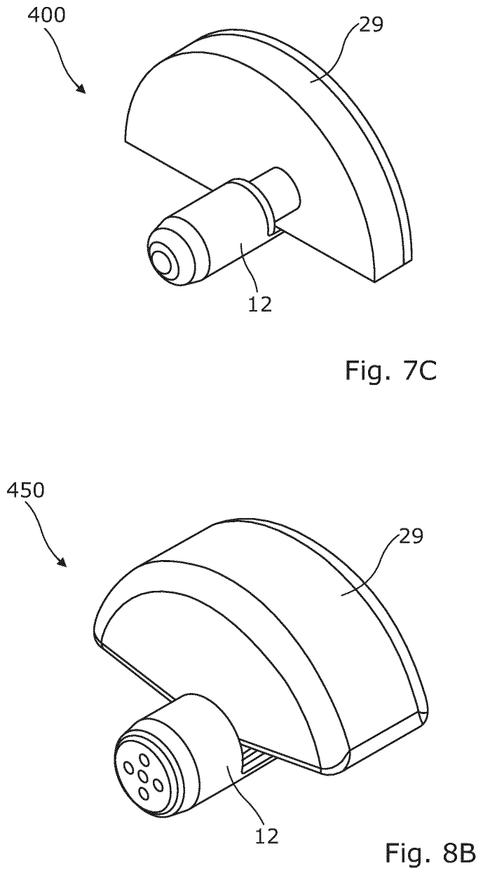

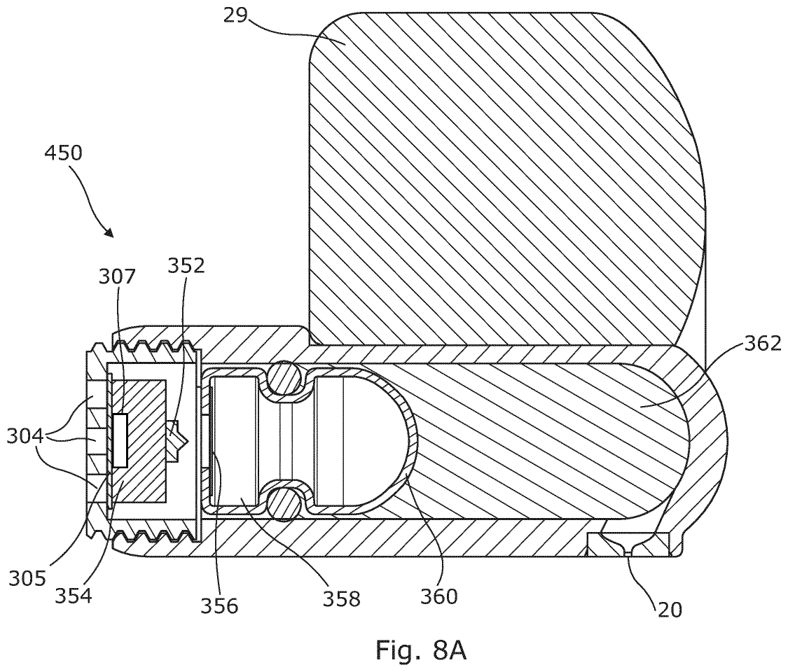

FIGS. 8a and 8b show a capsule device according to a ninth embodiment of the invention.

A capsule device 10 according to a first embodiment of the invention is shown in FIGS. 1a and 1b. The capsule device 10 is intended for oral administration and so is sized and shaped accordingly. In particular, the capsule device 10 includes a housing section 12 which is shaped as an elongated object, an oblong shape in this embodiment (although the invention is not restricted to this shape), which extends along an axis A. As shown in FIG. 1b, the elongate axis A is intended to extend along the same elongate axis of the intestinal tract 14 of a patient when the capsule device 10 is in the desired position for therapeutic substance delivery.

The housing section 12 defines an interior 16 that is configured to contain a therapeutic substance (not shown) in use, and has an exterior surface 18.

The capsule device 10 also includes a delivery outlet 20 that is positioned laterally to the axis A. The delivery outlet 20 may be an aperture to permit jet injection to occur. The capsule device 10 further includes a delivery assembly 22 that includes a jet injector (not shown) which is configured to deliver, in use, the therapeutic substance through the delivery outlet 20 and into the lumen wall 24 by jet injection.

Existing jet injector systems for jet drug delivery are known in the art. A skilled person would understand how to select an appropriate jet injector that provides the correct jetting power to deliver the therapeutic substance into the lumen wall 24, for example from WO 2020/106,750 (PROGENITY INC).

In particular, the skilled person would understand that during drug delivery into a GI tract of a patient using jet injection, the jet stream created by the jet injector interfaces the lumen of the GI tract and the surface of the GI tract facing the lumen. Ultimately, the drug substance is deposited into the submucosal and/or the mucosal tissue by the substance impacting the mucosal layer of the GI tract (e.g. the epithelial layer and any mucus that may be present on the epithelial layer) as a stable jet stream of fluid with minimal breakup into a spray.

The volume of fluid of the drug substance experiences a peak fluid pressure that generates the jet stream that exits the jet injector with a peak jet velocity. The jet stream impacts the interface of the lumen of the GI tract and the surface of the GI tract facing the lumen with a peak jet power, peak jet pressure and peak jet force. The skilled person would recognise that these three parameters are interconnected.

The skilled person would understand how to assess and measure the various jet injector characteristics for suitability of use in the described type of jet injection. For example, one way to assess the jet power is to release the jets onto force sensors which measure the force the jet. Based on the force reading, and knowing the area of the nozzle and density of the jetted liquid, the jet velocity can be determined using equation 1. Based on the calculated velocity, the power (in Watts) can be calculated using equation 2. To evaluate the jet pressure (i.e. the pressure at which the jet stream is expelled), equation 3 can be used.

$$F = ρ {AV}^{2} \qquad ( eqation 1 )$$

$$P = \frac{1}{2} ρ {AV}^{3} \qquad ( equation 2 )$$

$$V = \sqrt{\frac{2 * {P}_{bar} * 100000}{ρ * C}} \qquad ( equation 3 )$$

F=Force (N)
p=Density (kg/m3)
A=Area of nozzle (m2)
V=Velocity (m/s)
P=power (W)
P_{bar}=Pressure (bar)
C=Nozzle Loss Coefficient (Typically 0.95)

The capsule device 10 further includes an expansion assembly 26 that includes an expansion section 28. The expansion section 28 is arranged laterally opposite to and physically separate from the delivery outlet 20. The expansion section 28 is laterally expandable from a non-expanded configuration (as shown in FIG. 1a) to an expanded configuration (as shown in FIG. 1b). As illustrated in FIG. 1b, the expansion section 28 being in the expanded configuration orients the capsule device 10 within the tract 14 so that the delivery outlet 20 is positioned against the lumen wall 24.

Preferably, the expanded section 28 extends along the majority, or full, length of the housing section 12.

In this embodiment, the expansion section 28 is in the form of a sponge 29, which assumes the expanded configuration when wetted.

The capsule device 10 also includes an expansion control mechanism 30 which, in this embodiment, is in the form of an enteric coating 32. The coating 32 surrounds the whole capsule device 10, although this may not be the case in other embodiments, e.g. it may only cover the expansion section 28. The enteric coating 32 is configured to dissolve when it is exposed to the intestinal fluid in the tract 14 such that it exposes the expansion section 28, i.e. the sponge 29, to the intestinal fluid, thus activating expansion of the sponge 29.

The enteric coating 32 (sometimes referred to as a gastro-resistant coating) is a barrier that resists breakdown before it reaches the small intestine, and then dissolves due to the characteristics (e.g. pH, pressure, acidity, temperature, etc.) of the small intestine. The coating may take another form to dissolve in another part of the GI tract 14, depending on where substance delivery is required.

The expansion control mechanism 30 may be another form of dissolvable trigger such as a time-dependent coating which dissolves after a predetermined period of time. The expansion control mechanism 30 may include a sensor which detects a desired position within the GI tract 14 to then activate the expansion section 28.

Moreover, the expansion control mechanism 30 may include a combination of triggers to activate the expansion section 28.

In this embodiment, the sponge 29 is shown as a rectangular solid shape when expanded (although it may be a different solid shape such as an oval, circle or square). The sponge 29 in this embodiment is biodegradable such that is degrades over time, e.g. in nature.

The length of the expansion section 28 should be such the capsule device 10 will start to turn and align with the longitudinal axis of the lumen 14 when the expansion section 28 is triggered. Moreover, the overall width of the capsule device 10 when the expansion section 28 is in the expanded configuration should be such that it presses the delivery outlet 20 against the lumen wall 24, thus allowing injection to take place at the intended injection site. The expansion section 28, however, should not expand so much that it would cause great discomfort to the patient or damage the lumen wall in any way. The length of the expanded section 12 preferably should be equal to or larger than the diameter of the lumen 14. For example, it could be 2× maximum diameter of the lumen. However, there may be instances where the length of the expanded section 12 is less than the lumen diameter. The overall width of the device 10 with the expansion section 28 in the expanded configuration is preferably equal to or more than the maximum lumen diameter.

The capsule device 10 in this example has the following dimensions. The tablet housing 12 has a length of about 25 mm and a width of about 8.5 mm. The width of the expansion section in the expanded configuration is around 30 mm and the length in the expanded configuration is between 30-70 mm, preferably between 50-70 mm. The capsule device 10 may be configured as a 00-sized capsule or a 000-sized capsule.

It will be understood that these dimensions are for illustrational purposes only and any suitable dimensions may be chosen depending on the intended injection site and/or the patient in question. For example, the dimensions described above are intended for use in an adult small intestine, which typically has a dimeter of between 25 to 30 mm. However, a child's small intestine diameter is smaller and so different dimensions can be chosen (particularly for the width and length of the expanded section) depending on the patient's age bracket. Data regarding the dimensions of an intended injection site, e.g. the intestine, would be readily available to a skilled person.

The description in relation to the dimensions of the capsule device 10 and expanded section 28 applies to the other embodiments of the invention described below.

In use, the capsule device 10 is swallowed by a patient and moves along a lumen of the intestinal tract 14 of the patient. When the capsule device 10 reaches the small intestine tract, the enteric coating 32 begins to dissolve due to the conditions of the small intestine. Such dissolving results in the expansion section 28, i.e. the sponge 29, being exposed to the small intestine fluid.

The fluid wets the sponge 29 which causes the sponge 29 to expand from its non-expanded configuration to its expanded configuration. The size and positioning of the expanded sponge 29 relative to the size of the lumen causes the capsule device 10 to position itself longitudinally along the longitudinal axis of the lumen. In doing so, the delivery outlet 20 is positioned against the lumen wall 24, ready for jet injection by the delivery assembly 22.

Jet injection is then carried out by the jet injector to deliver the therapeutic substance into the lumen wall 24 of the patient.

The length of the capsule device 10 is just larger than the lumen diameter and/or the lumen diameter when in a contracted configuration (e.g. when no food is passing through) so that the capsule device 20 cannot orient itself vertically within the lumen (i.e. the capsule device longitudinal axis A cannot lie perpendicular to the longitudinal axis of the lumen). Therefore, a portion of the sponge 29 (which preferably extends along the majority or full length of the housing section 12, or extends beyond the length of the housing section 12), when expanded, will push against the lumen wall 24 so as to orient the capsule device 10 as desired (i.e. as shown in FIG. 1b).

The sponge 29 may be configured to break up into smaller sponge pieces under a predetermined condition so as to allow the sponge pieces to pass through the tract 14. The sponge 29 may also or instead break away from the housing section 12 of the capsule device 10. Such breaking up and/or breaking away might be caused by a trigger, e.g. an electronic trigger, or a time-based dissolvable glue at the attachment points (i.e. where the sponge pieces are attached to one another and/or where the sponge is attached to the housing section 12), or a coating which dissolves to expose another dissolvable component at the attachment points which dissolves on exposure to the intestinal fluid (the first coating could be given a thickness which means it dissolves after a sufficient amount of time to allow injection to take place). Moreover, the sponge 29 may break away from the housing section 12 and then be triggered to dissolve under conditions of another portion of the GI tract.

A capsule device 100 according to a second embodiment of the invention is shown in FIGS. 2a and 2b. The capsule device 100 shares similar features to that of the capsule device 10 according to the first embodiment of the invention, and identical features are indicated by the same reference numerals.

The capsule device 100 of the second embodiment differs from the capsule device 10 of the first embodiment in that the expansion section 28 is in the form of a sponge 102 which creates a passage 104 when it assumes the expanded configuration.

In particular, the sponge 102 is formed from an elongate section of sponge material which is attached only at each end 105 to opposing sides of the housing section 12 of the capsule device 100. This way, when the sponge 102 expands, the section of sponge 102 which is not attached to the housing section 12 is free to expand outwards and away from the housing section 12. Thus, essentially forming a closed loop that is defined by the sponge 102 and housing section 12. The hole of the loop therefore forms a passage 104.

The housing section and/or sponge 102 may include mutually abuttable or engageable portions to help with attachment of the sponge 102 to the housing section 12. In this embodiment, the housing section 12 includes a ledge 106 on each opposing side of the capsule device 100, each ledge 106 extending longitudinally along the exterior surface of the housing section 12. The ledges 106 may extend only partially or fully along the length of the device 100. The ends 106 of the sponge 102 nest within the ledges 106 where they are attached to the housing section 12.

Although not shown in FIG. 2a, the capsule device 100 includes an expansion control mechanism which exposes the sponge 102 to fluid to allow it to expand under a predetermined condition. As set out previously, this may be a dissolvable coating (such as an enteric coating) or may take any other suitable form.

In a similar manner as described above in relation to the first embodiment of the invention, the expansion of the sponge 102 orients the housing section 12 within the tract (not shown in FIG. 2b) so that the delivery outlet 20 is against the lumen wall, ready for injection. The looped sponge 102 presses slightly against the lumen wall to help position the capsule device 100. Meanwhile, the hole created by the looped sponge 102 forms a passage 104 for substance such as chyme to pass through, thus preventing blockages.

The sponge 102 may also be formed from biodegradable or otherwise dissolvable sponge material. In addition, or alternatively, the sponge 102 may be configured to break up into smaller sponge pieces or break away from the housing section 12 after a predetermined period of time so that it can pass safely through the tract. As outlined above in relation to the first embodiment, such breaking up or away may be caused by different triggers.

FIGS. 2a and 2b are schematic representations of the capsule device 100 and as such their dimensions and relative sizing of components may not reflect the capsule device 100 in reality.

For example, the sponge 102 and the passage 104 are shown as exaggerated in size compared to the housing section 12.

A capsule device 150 according to a third embodiment of the invention is shown in FIGS. 3a and 3b. The capsule device 150 shares similar features to that of the capsule device 10 according to the first embodiment of the invention, and identical features are indicated by the same reference numerals.

The capsule device 150 of the third embodiment of the invention differs from that of the first embodiment of the invention in that the expansion section 28 is in the form of a sponge 152 which has been pre-compressed when it is in the non-expanded configuration in such a way that it causes radial expansion of the sponge 152 when in the expanded configuration (as shown in FIG. 3b). By being pre-compressed, the sponge 152 is wetted when it is in its compressed state and when it dries it will keep that compressed shape. Then, when the sponge 152 is wetted again (e.g. by intestinal fluid) it will expand again.

The compressed sponge 152 may take the form of several (e.g. three to five) distinct sponge pieces, each of which is secured to the housing section 12 of the capsule device 150. The size and shape of the sponge pieces, as well as the pre-compression, helps to determine the radial nature of the expansion.

Although not shown in FIG. 3a, the capsule device 150 includes an expansion control mechanism which exposes the sponge 152 to fluid to allow it to expand under a predetermined condition. As set out previously, this may be a dissolvable coating (such as an enteric coating) or may take any other suitable form.

The sponge 152 may be configured to break up into smaller sponge pieces under a predetermined condition so as to allow the sponge pieces to pass through the tract 14. The sponge 152 may also or instead break away from the housing section 12 of the capsule device 150. Such breaking up and/or breaking away might be caused by a trigger, e.g. an electronic trigger, or a time-based dissolvable glue at the attachment points (i.e. where the sponge pieces are attached to one another and/or where the sponge is attached to the housing section 12), or a coating which dissolves to expose another dissolvable component at the attachment points which dissolves on exposure to the intestinal fluid (the first coating could be given a thickness which means it dissolves after a sufficient amount of time to allow injection to take place).

Moreover, the sponge 152 may break away from the housing section 12 and then be triggered to dissolve under conditions of another portion of the GI tract.

Operation of the capsule device 150 in use is similar to that already described above in relation to the first embodiment.

A capsule device 200 according to a fourth embodiment of the invention is shown in FIGS. 4a and 4b. The capsule device 200 shares similar features to that of the capsule device 10 according to the first embodiment of the invention, and identical features are indicated by the same reference numerals.

The capsule device 200 of the fourth embodiment of the invention differs from that of the first embodiment of the invention in that the expansion section 28 is in the form of an inflatable bag 202.

The inflatable bag 202 is folded onto itself when in the non-expanded configuration so as to provide a compact configuration, as shown in FIG. 4a. When the inflatable bag 202 is in the expanded configuration (as shown in FIG. 4b), it unfolds as it inflates due to it being filled with fluid (e.g. liquid or gas), thus orienting the capsule device 200 as desired.

The inflatable bag 202 may be made from a biodegradable material, e.g. polybutylene adipate terephthalate (PBAT) which is biodegradable and compostable.

The capsule device 200 further includes an effervescent reaction mechanism 204 that is configured to activate an effervescent reaction to inflate the inflatable bag 202 to assume the expanded configuration.

In this embodiment, the effervescent reaction mechanism 204 includes first and second chambers 206 which are separated by a dissolvable separator 208. The first and second chambers 206 contain components that, when mixed, cause the effervescent reaction.

The following are examples of chemical reactions which produce carbon dioxide CO₂ and which may be used as the components in the chambers 206:

### Example 1 (Calcium Carbonate with Hydrochloric Acid): CaCo3+2HCl→CaCl₂+H2O+CO2

### Example 2 (Citric Acid with Sodium Bicarbonate): C6H8O7+3NaHCO3→3H2O+CO2+Na3C6H5O7

### Example 3 (Tartaric Acid with Sodium Bicarbonate): H2C4H406+2NaHCO3→Na2C4H4O6+2H2O+2CO2

Examples of acids for effervescent reaction:
Citric acid
Acetic acid
Hydrochloric acid
Tartaric acid
Malic acid
Adipic acid
Ascorbic acid
Fumaric acid

Examples of carbonate salts for effervescent reaction:
Sodium bicarbonate
Sodium carbonate
Calcium carbonate
Potassium bicarbonate

In other embodiments, the effervescent reaction may occur by one or more solid state components being wetted (e.g. exposed to intestinal fluid or other fluid kept in the device 100) which causes the effervescent reaction.

The dissolvable separator 208, in this embodiment, is dissolved when it is exposed to the surrounding intestinal fluid. Such exposure occurs after the enteric coating 32 is dissolved.

It will be understood that the separator 108 may be triggered by any other suitable means, as already discussed in relation to the expansion control mechanism 30.

Moreover, the dissolvable separator 108 in this embodiment is a clamp 210 which pinches a joining portion 212 between the first and second chambers 206 to keep the components separate from one another. As the clamp 210 dissolves, it releases the joining portion 212 which creates a path between the first and second chambers 206 to allow the components to mix.

The clamp 210 can be formed in different sizes/thicknesses to control the timing of the effervescent reaction.

The capsule device 200 may further include a filter (not shown) positioned in the conduit between the chambers 206 and the bag 202. The filter ensures that powder from the reaction remains in the chambers 206 while letting the gas from the effervescent reaction to pass through to inflate the bag 202.

In use, as with the first embodiment of the invention, the capsule device 200 is swallowed by a patient and moves along a lumen of the intestinal tract 14 of the patient. When the capsule device 200 reaches the small intestine tract, the enteric coating 32 begins to dissolve due to the conditions of the small intestine. Such dissolving results in the dissolvable clamp 210 (which is part of the expansion assembly) being exposed to the conditions of the small intestine. This may occur because the enteric coating 32 reveals an aperture through the housing section 12 to the clamp 210.

The clamp 210 then dissolves due to the conditions of the small intestine. It may also be made from an enteric coating material which dissolves in the intestinal fluid. The dissolving of the clamp 210 releases the joining portion 212 of the chambers 206, which creates a path between the first and second chambers 206 to allow the components to mix. Such mixing causes the effervescent reaction to occur which, in turn, provides gas pressure to inflate the bag 202 to its expanded configuration.

The size and positioning of the inflated bag 202 relative to the size of the lumen causes the capsule device 200 to position itself longitudinally along the longitudinal axis of the lumen. In doing so, the delivery outlet 20 is positioned against the lumen wall, ready for jet injection by the delivery assembly 22.

Jet injection is then carried out by the jet injector to deliver the therapeutic substance into the lumen wall of the patient.

The bag 202 may be configured to break away from the housing section 12 under a predetermined condition so as to allow the bag 202 to pass through the tract. Such breaking away might be caused by a trigger, e.g. an electronic trigger, or a time-based dissolvable glue at the attachment points (i.e. where the bag 202 is attached to the housing section 12), or a coating which dissolves to expose another dissolvable component at the attachment points which dissolves on exposure to the intestinal fluid (the first coating could be given a thickness which means it dissolves after a sufficient amount of time to allow injection to take place). Moreover, the bag 202 may break away from the housing section 12 and then be triggered to dissolve under conditions of another portion of the GI tract.

A capsule device 250 according to a fifth embodiment of the invention is shown in FIGS. 5a and 5b. The capsule device 250 shares similar features to that of the capsule device 10 according to the first embodiment of the invention, and identical features are indicated by the same reference numerals.

The capsule device 250 of the fifth embodiment of the invention differs from that of the first embodiment of the invention in that the expansion section 28 is in the form of an inflatable bag 252 which contains a plurality of sponge pieces 254.

The inflatable bag 252 may be folded onto itself when in the non-expanded configuration, or may simply be deflated, so as to provide a compact configuration.

The inflatable bag 252 includes a plurality of holes 256 to permit exposure of the sponge pieces 254 to the intestinal fluid. Such exposure causes the sponge pieces 254 to swell, thus filling the bag 252 so that it assumes its expanded configuration.

The holes 256 are sized so that they are smaller than the size of the sponge pieces 254 so that the sponge pieces 254 do not escape through the holes 256. For example, the holes 256 may be 1 mm in diameter and the sponge pieces 254 may be 1.5 mm in diameter.

In this embodiment, the capsule device 250 includes the enteric coating (not shown), which dissolves in the small intestine thereby exposing the holes 256 (and thus the sponge pieces 254) to the intestinal fluid. The capsule device 250 may include another type of trigger, as discussed previously. Moreover, the sponge pieces 254 may be formed or coated in such a way that they only expand when exposed to the conditions of a particular part of the GI tract (e.g. the conditions in the small intestine).

Also, in this embodiment, the bag 252 is made up of two bag portions 252a, 252b that are secured together along a join line 258. The bag portions 252a, 252b are joined together by a dissolvable glue which is selected to that it dissolves under predetermined conditions, which results in dissolving taking place after jet injection has occurred. Once dissolved, it leaves the join line 258 open such that the sponge pieces 254 are released from the bag 252 and are free to move along the remainder of the GI tract.

As discussed previously, the dissolvable glue may dissolve due to exposure to the intestinal conditions and/or it may dissolve based on a period of time. In addition, or alternatively, the join line 258 may include another dissolvable coating which is exposes the dissolvable glue to allow it to dissolve in the intestinal fluid. In addition, or alternatively, the bag 252 may be configured to break away from the housing section 12, as discussed previously. In addition, or alternatively, the sponge pieces 254 may be configured to break up into smaller pieces (as discussed previously).

Examples of useful water-soluble polymers for the dissolvable glue include, but are not limited to, polyethylene oxide (PEO), pullulan, hydroxypropylmethyl cellulose (HPMC), hydroxyethyl cellulose (HPC), hydroxypropyl cellulose, polyvinyl pyrrolidone, carboxymethyl cellulose, polyvinyl alcohol, sodium aginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof.

Examples of degradable polymers for dissolvable glue include, but are not limited to: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof.

In other embodiments, the capsule device 250 further includes an effervescent reaction mechanism, as described previously in relation to the fourth embodiment. The bag 252 therefore assuming the inflated configuration due to a combination of the effervescent reaction and the sponge pieces 254.

A capsule device 300 according to a sixth embodiment of the invention is shown in FIG. 6a. The capsule device 300 shares similar features to that of the capsule device 200 according to the fourth embodiment of the invention, and identical features are indicated by the same reference numerals.

The capsule device 300 shown in FIG. 6a additionally shows a mechanism for initiating the jet injection. The capsule device 300 includes a sponge portion 302 which, after the enteric coating (not shown) dissolves, is exposed to the gastro fluid via openings 304 and begins to expand. A semi-permeable membrane 305 is positioned across the openings 304 on the inner side of the device 300. To enable the semi-permeable membrane 305 to quickly soak in gastric fluid through the openings 304, i.e. to serve in combination with the semi-permeable membrane as an osmotic drive, a salt 307 or similar material is positioned in contact with both the semipermeable membrane 305 and the sponge portion 302.

Swelling of the sponge portion 302 in the axial direction causes a neighbouring gas canister 306 to be forced to displace distally. Attached to the gas canister 306 at the opposing side of the sponge portion 302 is a rupturable seal 308. The rupturable seal 308 acting to contain the pressurised gas in the gas canister 306.

As the gas canister 306 moves the rupturable seal 308 is brought into contact with a spike 310 which is pointed in the direction of the rupturable seal 308. The spike 310 ruptures the seal 308, thus releasing the pressurised gas in the gas canister 306. The pressurised gas exerts a force onto a neighbouring piston 312, which causes the piston 312 to drive hydraulic pressure in the drug substance reservoir 314 to thrust the substance through the delivery outlet 20 to perform jet injection.

FIG. 6b shows a capsule device 350 according to a seventh embodiment of the invention in which an alternative example of a mechanism for initiating the jet injection is shown. This time, the spike 352 is secured to the sponge portion 354 and as the sponge portion 354 expands axially, it pushes the spike 352 towards the rupturable seal 356 of the gas canister 358. The spike 352 ruptures the seal 356, thus causing the pressurised gas to escape the gas canister 358. The pressurised gas exerts a force onto a neighbouring piston 360, which causes the piston 360 to drive hydraulic pressure in the drug substance reservoir 362 to thrust the substance through the delivery outlet 20 to perform jet injection.

FIGS. 7a, 7b and 7c show a capsule device 400 that is similar to that shown in FIG. 6a and the same features share the same reference numerals. The capsule device 400 differs in that it includes a sponge 29 as the expanded section. FIG. 7a shows the device 400 with the sponge 29 in the non-expanded configuration, and FIGS. 7b and 7c show the device 400 with the sponge 29 in the expanded configuration.

FIGS. 8a and 8b show a capsule device 450 that is similar to that shown in FIG. 6b and the same features share the same reference numerals. The capsule device 450 differs in the it includes a sponge 29 as the expanded section. FIGS. 8a and 8b show the sponge 29 in the expanded configuration.

It will be understood that the inflatable bag shown and described above may take the form of an inflatable balloon. The balloon may be made from materials such as rubber, latex, polychloroprene.

It will also be understood that where a sponge material is described in relation to the embodiments of the invention, another swellable component or substance could be used. For example, a swellable polymer or hydrogel could be used in combination with or instead of the sponge material.

1. A capsule device suitable for swallowing into a lumen of an intestinal tract of a patient, the lumen having a lumen wall, the capsule device comprising:
a housing section shaped as an elongated object extending along an axis, the housing section defining an interior and having an exterior surface, the interior being configured to contain a therapeutic substance in use,
a delivery outlet disposed at the housing section and laterally to the axis, the delivery outlet being shaped to allow needle-less jet injection of the therapeutic substance into the lumen wall,
wherein the capsule device further comprises an expansion assembly comprising an expansion section arranged laterally opposite to and physically separated from the delivery outlet, wherein the expansion section is laterally expandable from a non-expanded configuration to an expanded configuration for positioning the delivery outlet against the lumen wall, and
wherein the capsule device comprises an expansion control mechanism configured to activate the expansion assembly under a predetermined condition to permit change of the expansion section from the non-expanded configuration to the expanded configuration.

2. The capsule device as in claim 1, wherein the expansion section comprises an inflatable balloon, wherein the balloon assumes a non-inflated configuration when the expansion section assumes the non-expanded configuration and assumes an inflated configuration when the expansion section assumes the expanded configuration.

3. The capsule device as in claim 2, wherein the inflatable balloon forms an inflatable bag, wherein the bag assumes a folded configuration when the expansion section assumes the non-expanded configuration and assumes an inflated configuration when the expansion section assumes the expanded configuration.

4. The capsule device as in claim 1, wherein the expansion section comprises a swellable component or substance configured to expand when exposed to gastrointestinal fluid.

5. The capsule device as in claim 4, wherein the swellable component or substance comprises a hydrogel configured to expand from the non-expanded configuration to the expanded configuration when exposed to gastrointestinal fluid.

6. The capsule device as in claim 4, wherein the swellable component or substance comprises a sponge material configured to expand from the non-expanded configuration to the expanded configuration when exposed to gastrointestinal fluid.

7. The capsule device as in claim 6, wherein the sponge material, when in the expanded configuration, creates a passage therethrough.

8. The capsule device as in claim 2, claim 2 or 3, wherein the inflatable balloon contains a plurality of swellable components or substances configured to expand when exposed to fluid.

9. The capsule device as in claim 8, wherein the inflatable balloon includes at least one aperture to permit exposure of the swellable components or substances to gastrointestinal fluid.

10. The capsule device as in claim 8, wherein the inflatable balloon is configured to release the swellable component or substances after a predetermined time.

11. The capsule device as in claim 2, wherein the expansion control mechanism includes an effervescent reaction mechanism configured to activate an effervescent reaction to inflate the balloon to assume the expanded configuration.

12. The capsule device as in claim 11, wherein the expansion control mechanism includes a separator which separates components that, when mixed, cause the effervescent reaction, the separator being configured to dissolve when exposed to gastrointestinal fluid to allow the components to mix.

13. The capsule device as in claim 1, wherein the expansion section is configured to break up into separate expansion section pieces after a predefined amount of time of being exposed to gastrointestinal fluid.

14. The capsule device as claimed in claim 1, wherein the expansion section is configured to break away from the housing section after a predefined amount of time of being exposed to gastrointestinal fluid.

15. The capsule device as in claim 1, wherein the expansion control mechanism includes a coating configured to dissolve when exposed to gastrointestinal fluid.
